(19) United States
(12) Patent Application Publication
(10) Pub. No.: US 2020/0100691 A1
(43) Pub. Date: Apr. 2, 2020

(54) NEUROMONITORING DEVICE

(71) Applicant: JIANGSU BAINING YINGCHUANG MEDICAL TECHNOLOGY CO., LTD., Nanjing (CN)

(72) Inventors: Zhigang SHI, Nanjing (CN); Jiancong LI, Nanjing (CN)

(73) Assignee: JIANGSU BAINING YINGCHUANG MEDICAL TECHNOLOGY CO., LTD., Nanjing (CN)

(21) Appl. No.: 16/699,847

(22) Filed: Dec. 2, 2019

Related U.S. Application Data
(63) Continuation of application No. PCT/CN2019/086104, filed on May 9, 2019.

(30) Foreign Application Priority Data
Aug. 1, 2018 (CN) 201810863181.9

(51) Int. Cl. A61B 5/04; A61B 5/00

(52) U.S. Cl. CPC A61B 5/04001; A61B 5/7475; A61B 5/742

(57) ABSTRACT

The present disclosure relates to a neuromonitoring device. The device includes a probe, a handle, and an elastic prompt. The probe is connected to the handle; the probe includes a probe head, an elastic piece, and an elastic measuring piece; the probe head is connected to the elastic piece; the elastic measuring piece is connected to the elastic piece and is used to measure an elasticity value of the elastic piece and convert the elasticity value into an electrical signal; the elastic prompt is electrically connected to the elastic measuring piece and is used to receive the electrical signal and generate prompt information regarding the elasticity value based on the electrical signal.

### CROSS-REFERENCE TO RELATED APPLICATIONS

This application is a continuation of International Application No. PCT/CN2019/086104, filed on May 9, 2019, which claims priority of Chinese Patent Application No. 201810863181.9, entitled “A NEUROMONITORING PROBE FOR SENSING CONTACT STATE,” filed on Aug. 1, 2018, the entire contents of which are incorporated herein by reference.

### TECHNICAL FIELD

The present disclosure relates to the field of medical devices, and in particular, to a neuromonitoring device.

### BACKGROUND

Neuromonitoring probes are often used during surgery. The neuromonitoring probe is usually connected to a neuro monitor and a surgeon uses the neuromonitoring probe to locate and identify nerves at risk in a surgical area, thus nerves can be protected from injury during the operations. For existing neuromonitoring probes, there may be some problems such as inconvenience in operation, difficulty in controlling the strength and the magnitude of the stimulation current, etc. Therefore, it is desirable to provide an improved neuromonitoring device.

### SUMMARY

The present disclosure provides a neuromonitoring device, the device includes a probe, a handle, and an elastic prompt; the probe is connected to the handle; the probe includes a probe head, an elastic piece, and an elastic measuring piece; the probe head is connected to the elastic piece; the elastic measuring piece is connected to the elastic piece and is used to measure an elasticity value of the elastic piece and convert the elasticity value into an electrical signal; the elastic prompt is electrically connected to the elastic measuring piece and is used to receive the electrical signal and generate prompt information regarding the elasticity value based on the electrical signal.

In some embodiments, the elastic prompt is set on the handle.

In some embodiments, the elastic prompt is used to display the elasticity value.

In some embodiments, the neuromonitoring device further includes an elastic adjustment part used to adjust a maximum elasticity value of the elastic piece.

In some embodiments, the elastic piece is made of a conductive material.

In some embodiments, the handle is provided with a current adjustment part used to adjust a magnitude of a nerve stimulation current.

In some embodiments, the neuromonitoring device further includes a monitor, and the monitor includes: a host used to receive a current adjustment signal sent by the current adjustment part and generate a current control signal; a current output unit used to receive the current control signal generated by the host and output a current of a corresponding magnitude to the probe.

In some embodiments, the current control signal includes a pulse width modulation wave control signal.

In some embodiments, the current adjustment part includes at least one button.

In some embodiments, the current adjustment part is further used to: adjust the magnitude of the nerve stimulation current based on a first adjustment step size within a first current value range; adjust the magnitude of the nerve stimulation current based on a second adjustment step size within a second current value range.

In some embodiments, the handle is provided with a current display part used to display a magnitude of a nerve stimulation current.

In some embodiments, the neuromonitoring device further includes a probe monitoring part used to monitor a usage status of the probe and generate probe monitoring information, wherein the usage status of the probe includes a cumulative usage time of the probe and/or an elastic condition of the elastic piece.

In some embodiments, the probe further includes a sleeve, the elastic piece is installed in the sleeve, an end of the probe head is inserted into a first end of the sleeve to connect to the elastic piece and a second end of the sleeve is connected to the handle.

In some embodiments, the end of the probe head inserted into the sleeve is provided with a non-slip step and an inner wall of the sleeve is provided with a limit step matching the non-slip step.

In some embodiments, a surface of the sleeve is provided with an insulation layer.

In some embodiments, an end of the probe head in contact with a human body is a ball-head structure.

### BRIEF DESCRIPTION OF THE DRAWINGS

The present disclosure is further described in terms of exemplary embodiments. These exemplary embodiments are described in detail with reference to the drawings. These embodiments are not limiting, and in these embodiments, like reference numerals represent similar structures, and wherein:

FIG. 1 is a schematic diagram illustrating a profile of a neuromonitoring device according to some embodiments of the present disclosure;

FIG. 2 is a schematic diagram illustrating a structure of a neuromonitoring device according to some embodiments of the present disclosure; and

FIG. 3 is a schematic diagram illustrating a connection structure of a probe head and a sleeve according to some embodiments of the present disclosure.

### DETAILED DESCRIPTION

In order to illustrate the technical solutions related to the embodiments of the present disclosure, a brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless apparent from the locale or otherwise stated, like reference numerals represent similar structures or operations throughout the several views of the drawings.

As used in the disclosure and the appended claims, the singular forms “a,” “an,” and “the” include plural forms as well unless the content clearly indicates otherwise. In general, the terms “comprise,” “comprising,” “include,” and/or “including” when used in this disclosure, specify the presence of stated steps and elements, and these steps and elements do not constitute an exclusive listing. The methods or devices may also include other steps or elements.

Although the present disclosure makes various references to certain modules in the system according to some embodiments of the present disclosure, any number of different modules may be used and run on a client terminal and/or a server. The modules are merely illustrative, and different aspects of the system and method may be implemented by different modules.

The flowcharts used in the present disclosure illustrate operations that systems implement according to some embodiments of the present disclosure. It should be understood that the preceding or following operations may be implemented not in order. Conversely, the operations may be implemented in an inverted order, or simultaneously. Moreover, one or more other operations may be added to the flowcharts. One or more operations may be removed from the flowcharts.

In order to illustrate the technical solutions related to the embodiments of the present disclosure, a brief introduction of the drawings referred to the description of the embodiments is provided below. Obviously, drawings described below are only some illustrations or embodiments of the present disclosure. Those having ordinary skills in the art, without further creative effort, may apply the present teachings to other scenarios according to these drawings. Unless apparent from the locale or otherwise stated, like reference numerals represent similar structures or operations throughout the several views of the drawings.

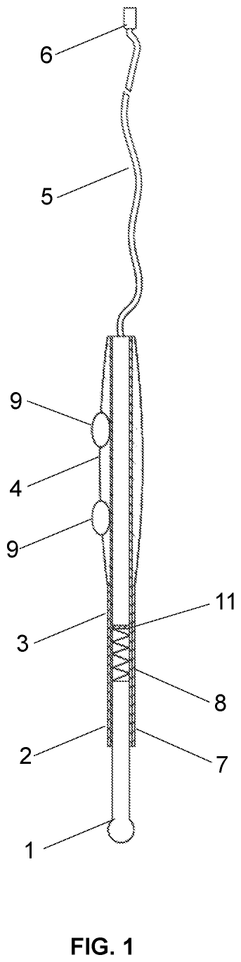

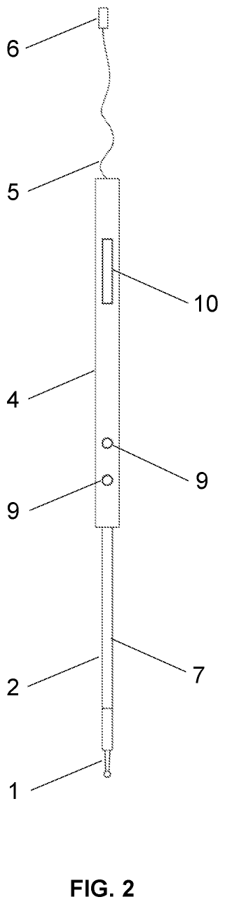

FIG. 1 is a schematic diagram illustrating a profile of a neuromonitoring device according to some embodiments of the present disclosure. FIG. 2 is a schematic diagram illustrating a structure of a neuromonitoring device according to some embodiments of the present disclosure. The neuromonitoring device may include a handle 4, a probe 7, and an elastic prompt 10. The probe 7 may be connected to the handle 4. The probe 7 may include a probe head 1, an elastic piece 8, and an elastic measuring piece 11. The probe head 1 may be connected to the elastic piece 8. When the neuromonitoring device is being used, the probe head 1 may contact with a human body (e.g., nerves, tissues) and may receive a pressure given by the human body. Further, the probe head 1 may transmit the pressure to the elastic piece 8 and then the elastic piece 8 may be elastically deformed, causing the probe head 1 to move. The probe head 1 is retractable due to the elastic deformation of the elastic piece 8, so that it can contact with the human body continuously and reliably. In addition, when using the neuromonitoring device of the present disclosure, a user may sense a resilience force and then sense a pressure exerted by the probe 7 on the human body, so that the user can control an operation strength of using the neuromonitoring device to ensure a reliable contact between the probe 7 and the nerves or the tissues. The elastic measuring piece 11 may be connected to the elastic piece 8 and may be used to measure an elasticity value of the elastic piece 8 and convert the elasticity value into an electrical signal. The elastic prompt 10 may be connected to the elastic measuring piece 11 and may be used to receive the electrical signal regarding the elasticity value of the elastic piece 8 generated by the elastic measuring piece 11 and generate prompt information regarding the elasticity value of the elastic piece 8 based on the electrical signal. The elastic prompt 10 may prompt the elasticity value of the elastic piece 8 in various forms, including but not limited to texts, images, voices, etc.

As illustrated in FIG. 2, the elastic prompt 10 may be set on the handle 4. In some embodiments, the elastic prompt 10 may include a display screen used to display the elasticity value. In some embodiments, when the elasticity value exceeds a set threshold, the elastic prompt 10 may provide an alert (e.g., displaying a warning image, providing a warning tone) to remind the user to control an operation strength. The set threshold may be a fixed value or may be determined based on different kinds of nerves to be detected. Merely for example, for cranial nerves, since the cranial nerves are relatively sensitive, the threshold may be set as a relatively low value (e.g., 0.8 N); for laryngeal nerves, the threshold may be set as 1.2 N; for nerves at a face, a hand, a foot, or a knee, the threshold may be set as 3 N.

In some embodiments, the neuromonitoring device of the present disclosure may further include a monitor (not shown). In some embodiments, one end of a wire 5 may be connected to the probe 7 and the other end may be connected to the monitor through a socket 6. In some embodiments, the elastic prompt 10 may be set in the monitor. Specifically, the monitor may receive the electrical signal regarding the elasticity value of the elastic piece 8 generated by the elastic measuring piece 11 and generate prompt information regarding the elasticity value of the elastic piece 8. For example, the monitor may include a display screen through which the elasticity value may be displayed. In addition to a text display, the monitor may also prompt the elasticity value by means of images, voices, etc. Since the elastic prompt 10 is used, when using the neuromonitoring device of present disclosure, the user (e.g., a doctor) can conveniently know a pressure applied to a patient and then control the operation strength, which can ensure a reliable contact between the probe 7 and the nerves or the tissues, and also can protect the nerves or the tissues of the patient from injury.

In some embodiments, different types of neuromonitoring devices may have different maximum elasticity values. For example, elastic pieces with different elastic coefficients may be used to achieve the differentiation of the maximum elasticity value. Specifically, according to Hooke's law:

F=k×X (1)

where F refers to an elasticity value of an elastic piece, k refers to an elastic coefficient of the elastic piece, and X refers to an elastic deformation of the elastic piece. According to equation (1), for elastic pieces with different elastic coefficients k, when a same elastic deformation X occurs, elasticity values F are different. Accordingly, in a situation that the maximum elastic deformation is fixed, different maximum elasticity values can be achieved by selecting elastic pieces with different elastic coefficients. In some embodiments, neuromonitoring devices with different maximum elasticity values may be used for different types of surgery. For example, for nerves with a relatively high sensitivity, a neuromonitoring device with a relatively small maximum elasticity value may be used; for nerves with a relatively low sensitivity, a neuromonitoring device with a relatively high maximum elasticity value may be used. Merely for example, for cranial nerves, a neuromonitoring device with a maximum elasticity value of 0.8 N may be used; for laryngeal nerves, a neuromonitoring device with a maximum elasticity value of 1.2 N may be used; for nerves at a face, a hand, a foot, or a knee, a neuromonitoring device with a maximum elasticity value of 3 N may be used. In some embodiments, neuromonitoring devices with different maximum elasticity values may be used for different individuals. For example, for patients with a relatively high sensitivity, a neuromonitoring device with a relatively small maximum elasticity value may be used; for patients with a relatively low sensitivity, a neuromonitoring device with a relatively high maximum elasticity value may be used.

The elastic measuring piece 11 may convert the elasticity value of the elastic piece 8 into an electrical signal. In some embodiments, the elastic measuring piece 11 may include an adjustable resistor connected to the elastic piece 8, whose resistance may change with a change of a length of the elastic piece 8, thereby realizing the conversion of the elasticity value to the electrical signal. For example, the elasticity value may be positively related to the resistance value; or the elasticity value may be inversely related to the resistance value. In some embodiments, the elastic measuring piece 11 may include a pressure sensor which may measure the elasticity value of the elastic piece 8. Specifically, when the neuromonitoring device is being used, when the probe head 1 is in contact with the human body and receives the pressure given by the human body, the elastic piece 8 may be compressively deformed and exert a pressure on the pressure sensor, then the elasticity value of elastic piece 8 may be obtained based on a pressure value measured by the pressure sensor.

In some embodiments, the elastic piece 8 may be also connected to an elastic adjustment part (not shown) used to adjust the maximum elasticity value of the elastic piece 8. For example, the maximum elasticity value may be adjusted and an elastic force may be changed by limiting the stretchable length of the elastic piece 8. For different types of surgery, the maximum elasticity value of the elastic piece 8 may be adjusted by the elastic adjustment part to match a maximum elasticity value corresponding to a type of surgery. For example, for cranial nerves, the maximum elasticity value of elastic piece 8 may be adjusted to 0.8 N; for laryngeal nerves, the maximum elasticity value may be adjusted to 1.2 N; for nerves at a face, a hand, a foot, or a knee, the maximum elasticity value may be adjusted to 3 N.

In some embodiments, the elastic piece 8 may be made of a conductive material. The conductive material may include a metal, a conductive rubber, a conductive non-metal, a conductive alloy, or the like, or a combination thereof. In some embodiments, the maximum elasticity value of the elastic piece 8 may be also adjusted for different individuals. For example, for patients with a relatively high sensitivity, the maximum elasticity value may be decreased; for patients with a relatively low sensitivity, the maximum elasticity value may be increased.

In some embodiments, the handle 4 may be also provided with a current adjustment part 9 used to regulate a magnitude of a nerve stimulation current. In some embodiments, the current adjustment part 9 may be electrically connected to the monitor through a wire. After receiving a current adjustment signal sent by the current adjustment part 9, the monitor may control the magnitude of the output stimulation current. For example, the monitor may include a host and a current output unit. The host may be used to receive the current adjustment signal sent by the current adjustment part 9, generate a current control signal based on the current adjustment signal, and send the current control signal to the current output unit. The current output unit may output a current of a corresponding magnitude based on the received current control signal. In some embodiments, the current output unit may include a voltage/current conversion integrated circuit which can convert an input voltage into an output current. Specifically, after the host of the monitor receives the current adjustment signal, a microcontroller unit (MCU) of the host may control the input voltage of the voltage/current conversion integrated circuit by controlling a pulse width modulation (PWM) wave. The voltage/current conversion of the integrated circuit may output a current with an appropriate magnitude.

In some embodiments, for different types of nerves, stimulation currents of different magnitudes may be obtained by adjustment. For example, for cranial nerves, the stimulation current may be adjusted to 0˜0.5 mA; for laryngeal nerves, the stimulation current may be adjusted to 0.5 mA˜10 mA; for nerves at a face, a hand, a foot, or a knee, the stimulation current may be adjusted to 10 mA˜30 mA. In some embodiments, due to differences in sensitivity of different individuals, the stimulation currents of different magnitudes may be obtained by adjustment for different individuals. For example, for patients with a relatively high sensitivity, the stimulation current may be decreased; for patients with a relatively low sensitivity, the stimulation current may be increased.

In some embodiments, a maximum current threshold may be set to limit the stimulation current from exceeding the maximum current threshold, thereby ensuring the safety of detecting nerves or tissues. For example, the maximum current threshold may be 40 mA, 35 mA, 30 mA, 25 mA, 20 mA, etc. In some embodiments, different maximum current thresholds may be set for different types of nerves. For example, for cranial nerves, the maximum current threshold may be set as 0.5 mA; for laryngeal nerves, the maximum current threshold may be set as 10 mA; for nerves at a face, a hand, a foot, or a knee, the maximum current threshold may be set as 30 mA. In some embodiments, different maximum current thresholds may be set for different individuals. For example, for patients with a relatively high sensitivity, the maximum current threshold may be set as a relatively low value; for patients with a relatively low sensitivity, the maximum current threshold may be set as a relatively high value.

The current adjustment part 9 may be in various forms including but not limited to a button, a knob, a touch key, etc. In some embodiments, as illustrated in FIG. 1 and FIG. 2, the current adjustment part 9 may be two buttons used to increase and decrease the current respectively. An adjustment step size may be a fixed value or a changing value. In some embodiments, different adjustment step sizes may be set for different stimulation current ranges. It can be understood that for a relatively small stimulation current, an adjustment precision requirement is relatively high so that a relatively small adjustment step size may be set to achieve a high-precision adjustment; for a relatively large stimulation current, the adjustment precision requirement is relatively low so that a relatively large step size may be set to achieve a rapid adjustment. For example, for a range from 0 to 0.5 mA, the adjustment step size may be 0.01 mA; for a range from 0.5 mA to 1 mA, the adjustment step size may be 0.1 mA; in the range of 1 mA to 10 mA, the adjustment step size may be 0.5 mA; for a range from 10 mA to 30 mA, the adjustment step size may be 1 mA. It should be noted that the two buttons illustrated in FIG. 1 and FIG. 2 are an example of the current adjustment part, and are not intended to limit the present disclosure. In some embodiments, current adjustment parts of other forms may be set. For example, four buttons may be set, two of which are used to roughly adjust (increase or decrease) the stimulation current based on a first step size, and the other two are used to finely adjust the stimulation current based on a second step size, wherein the second step size is less than the first step size.

In some embodiments, the neuromonitoring device of the present disclosure may also include a stimulation current prompt used to prompt the magnitude of the stimulation current. The magnitude of the stimulation current may be prompted in various forms including but not limited to texts, images, voices, etc. In some embodiments, the stimulation current prompt may be set on the handle 4. For example, a display screen may be set on the handle 4 and may be used to display the magnitude of stimulation current. In some embodiments, the stimulation current prompt and the elasticity value prompt described above may be integrated as a same component; or both may be separate components. In some embodiments, the stimulation current prompt may be set on the monitor. For example, the display screen of the monitor may display the magnitude of stimulation current.

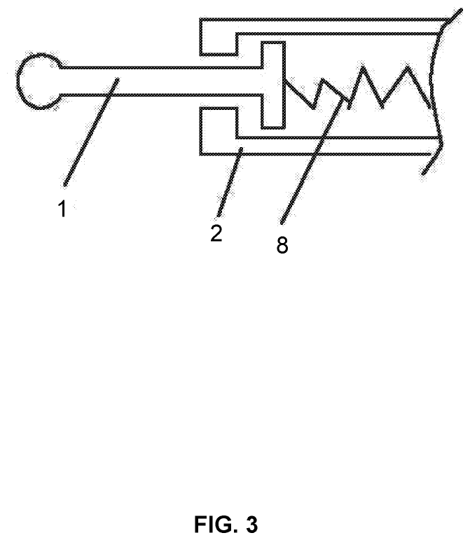

In some embodiments, the probe 7 may also include a sleeve 2. FIG. 3 is a schematic diagram illustrating a connection structure of a probe head 1 and a sleeve 2 according to some embodiments of the present disclosure. As illustrated in FIG. 1 and FIG. 3, the elastic piece 8 may be installed in the sleeve 2. One end of the probe head 7 may be inserted into a first end of the sleeve 2 to connect to the elastic piece 8 and a second end of the sleeve 2 may be connected to the handle 4. In some embodiments, the sleeve 2 may be made of a conductive material and the wire 5 may be electrically connected to the sleeve 2, thereby achieving an electrical connection between the wire 5 and the probe 7. In some embodiments, a surface of the sleeve 2 may be provided with an insulation layer 3 which may be a structure such as a heat shrinking sleeve, an insulating coating, etc. In some embodiments, the probe head 1 may be a ball-head structure. In some embodiments, in order to prevent the probe head 1 from slipping out of the sleeve 2, in addition to a manner that the probe head 1 is welded to the elastic piece 8, a non-slip step may be provided at one end of the probe head 1 inserted into the sleeve 2 and a matching limit step is provided on an inner wall of the sleeve 2. At the time of installation, the probe head 1 may be inserted into the sleeve 2 from the other end of the sleeve 2, and after the end of the probe head 1 provided with the step is in contact with the step inside the sleeve 2, the head of the probe head 1 may be spherically roughened. In addition, after the end of the probe head 1 provided with the step is inserted into the sleeve 2, an end portion of the sleeve 2 may be turned inward to form an inside step.

In some embodiments, the neuromonitoring device of the present disclosure may also include a probe monitoring part (not shown) used to monitor a usage status of the probe 7 and generate probe monitoring information. For example, the probe monitoring part may monitor a cumulative usage time of the probe. Merely for example, the probe monitoring part may read/write the cumulative usage time of the probe by an electrically erasable programmable read only memory (EEPROM). As another example, the probe monitoring part may monitor an elastic condition of the elastic piece in the probe. In some embodiments, in response to that the probe monitoring information satisfies a set condition, the probe monitoring part may provide a prompt. For example, when the cumulative usage time exceeds a certain time period or the elastic condition of the elastic piece decays to a certain extent, the probe monitoring part may provide an alarm to prompt the user to replace the elastic piece in time. In some embodiments, the probe monitoring part may be set on the handle 4. In some embodiments, the probe monitoring part may be integrated into the monitor.

The advantage effects of the embodiments of the present disclosure may include but not limited to: (1) an elastic piece is set to make a probe head retractable, which can ensure a reliable contact between the probe head and nerves or tissues; (2) the elastic piece also allows a user to sense a resilience force, in combination with an elastic prompt which can prompt an elasticity value, the user can know a pressure applied to a patient by the probe head during operation so as to adjust a strength in time to further ensure the reliable contact between the probe head and the nerves or the tissues and protect the nerves or the tissues from injury; (3) for different types of nerves or tissues, or for individuals with different sensitivities, neuromonitoring devices with different maximum elasticity values may be used, or appropriate maximum elasticity values may be adjusted, which can ensure the nerves or the tissues are not damaged by excessive pressures exerted by the probe under a premise of ensuring a detection effect; (4) for different types of nerves or tissues, or for individuals with different sensitivities, the magnitude of the stimulation current may be adjusted to achieve a better detection effect. It should be noted that different embodiments may have different advantage effects. In different embodiments, the advantage effects may include any combination of one or more of the above or any other possible advantage effect.

The basic concept has been described above, and it is obvious to those skilled in the art that the detailed disclosure is merely exemplary and does not constitute a limitation of the present disclosure. Various alterations, improvements, and modifications to the present disclosure may be made by those skilled in the art, although not explicitly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, certain features, structures, or features of one or more embodiments of the present disclosure may be combined as appropriate.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure method does not mean that the present disclosure object requires more features than the features mentioned in the claims. Rather, claim subject matter lie in less than all features of a single foregoing disclosed embodiment.

1. A neuromonitoring device, comprising a probe, a handle, and an elastic prompt; wherein
the probe is connected to the handle;
the probe includes a probe head, an elastic piece, and an elastic measuring piece; the probe head is connected to the elastic piece; the elastic measuring piece is connected to the elastic piece and is used to measure an elasticity value of the elastic piece and convert the elasticity value into an electrical signal;
the elastic prompt is electrically connected to the elastic measuring piece and is used to receive the electrical signal and generate prompt information regarding the elasticity value based on the electrical signal.

2. The neuromonitoring device of claim 1, wherein the elastic prompt is set on the handle.

3. The neuromonitoring device of claim 1, wherein the elastic prompt is used to display the prompt information regarding the elasticity value.

4. The neuromonitoring device of claim 1, further comprising an elastic adjustment part used to adjust a maximum elasticity value of the elastic piece.

5. The neuromonitoring device of claim 1, wherein the elastic piece is made of a conductive material.

6. The neuromonitoring device of claim 1, wherein the handle is provided with a current adjustment part used to adjust a magnitude of a nerve stimulation current.

7. The neuromonitoring device of claim 6, further comprising a monitor, wherein the monitor includes:
a host used to receive a current adjustment signal sent by the current adjustment part and generate a current control signal;
a current output unit used to receive the current control signal generated by the host and output a current of a corresponding magnitude to the probe.

8. The neuromonitoring device of claim 7, wherein the current control signal includes a pulse width modulation wave control signal.

9. The neuromonitoring device of claim 6, wherein the current adjustment part includes at least one button.

10. The neuromonitoring device of claim 6, wherein the current adjustment part is further used to:
adjust the magnitude of the nerve stimulation current based on a first adjustment step size within a first current value range; and
adjust the magnitude of the nerve stimulation current based on a second adjustment step size within a second current value range.

11. The neuromonitoring device of claim 1, wherein the handle is provided with a current display part used to display a magnitude of a nerve stimulation current.

12. The neuromonitoring device of claim 1, further comprising a probe monitoring part used to monitor a usage status of the probe and generate probe monitoring information, wherein the usage status of the probe includes at least one of a cumulative usage time of the probe or an elastic condition of the elastic piece.

13. The neuromonitoring device of claim 1, wherein
the probe further includes a sleeve,
the elastic piece is installed in the sleeve,
an end of the probe head is inserted into a first end of the sleeve to connect to the elastic piece and a second end of the sleeve is connected to the handle.

14. The neuromonitoring device of claim 13, wherein the end of the probe head inserted into the sleeve is provided with a non-slip step and an inner wall of the sleeve is provided with a limit step matching the non-slip step.

15. The neuromonitoring device of claim 13, wherein a surface of the sleeve is provided with an insulation layer.

16. The neuromonitoring device of claim 1, wherein an end of the probe head in contact with a human body is a ball-head structure.

17. A neuromonitoring probe, comprising: a handle, a wire, and a probe, wherein the probe includes a sleeve, a probe head, and an elastic piece, one end of the probe head is inserted into the sleeve and is electrically connected to the elastic piece installed in the sleeve, the elastic piece is positioned in and electrically connected to the sleeve, the elastic piece causes the probe head to generate a resilience force that can be sensed by a user; the handle is installed on the other end of the sleeve without the probe head installed, one end of the wire is electrically connected to the sleeve and the other end is connected to a nerve monitor.

18. The neuromonitoring probe of claim 17, wherein the elastic piece is a conductive elastic piece.

19. The neuromonitoring probe of claim 17, wherein the elastic piece is a metal spring, a conductive rubber part, a conductive non-metal spring, or a conductive alloy spring.

20. The neuromonitoring probe of claim 17, wherein an elasticity of the elastic piece is adjustable.

21-24. (canceled)
